(19) United States
(12) Patent Application Publication
(10) Pub. No.: US 2026/0144832 A1
(43) Pub. Date: May 28, 2026

(54) METHOD FOR PREVENTING OR TREATING CHRONIC PAIN

(71) Applicant: BENED BIOMEDICAL CO., LTD., Taipei City (TW)

(72) Inventors: Ying-Chieh TSAI, Taipei City (TW); Chien-Chen WU, Taipei City (TW); Fu-Sheng DENG, Taipei City (TW); Chin-Lin HUANG, Taipei City (TW); Chih-Chieh HSU, Taipei City (TW)

(21) Appl. No.: 18/962,154

(22) Filed: Nov. 27, 2024

(51) Int. Cl. A61K 35/747; A61K 35/00; A61P 25/02; C12N 1/20; C12R 1/225

(52) U.S. Cl. CPC A61K 35/747; A61P 25/00; C12N 1/205; A61K 2035/115; C12R 2001/225

(57) ABSTRACT

Provided are methods for preventing or treating a chronic pain in a subject that includes administering an effective amount of a composition of Lactococcus lactis DSM 27109 to the subject.

### BACKGROUND

### Technical Field

The present disclosure relates to a lactic acid bacterium, and more particularly relates to a lactic acid bacterium strain for prophylaxis or treatment of a chronic pain in a subject in need thereof.

### Description of Related Art

Chronic pain affects more people in more aspects than expected. Prevalence of chronic pain can be as high as 40%, and various manifestations of chronic pain, such as back pain, musculoskeletal disorders and neck pain, are the leading causes of years lost to disability. The personal, social and economic burden associated with chronic pain is enormous.

Given its multifaceted characteristics, it has been difficult to adequately define chronic pain. Recently, the International Association for the Study of Pain (IASP) has expanded the definition of chronic pain and defines the pain as “an unpleasant sensory and emotional experience associated with, or resembling that associated with, actual or potential tissue damage.” Hence, chronic pain is acknowledged as a pain that can occur in the absence of identifiable tissue damage. In addition, as pain is always subjective, a patient's report of pain should be accepted at face value in the absence of evidence to the contrary, although physicians might consider other means (e.g., facial expressions and imaging) to evaluate pain and identify causes.

While in practice there is considerable overlap in different types of pain mechanisms within and between patients, and pain classification is often considered as a continuum, there are now three recognized categories of chronic pain, including nociceptive (from tissue injury), neuropathic (from nerve injury), or nociplastic (from a sensitized nervous system). Among these, nociplastic pain is the latest classification of chronic pain. Previously, chronic pain was thought to arise via two sources: nociceptive, which is associated with an ongoing input from real or threatened tissue injury, and neuropathic, caused by injury or disease affecting the peripheral nervous system or central nervous system (CNS). However, there remain many other chronic pain conditions with well-defined phenotypes but without clear evidence of nociceptive or neuropathic involvement. In 2016, the term nociplastic pain was proposed as a mechanistic descriptor for chronic pain states not characterized by obvious activation of nociceptors or neuropathy, but in whom clinical and psychophysical findings suggest altered nociceptive function.

Nociplastic pain represents a dynamic interplay of various mechanisms causing or amplifying pain, arising de novo or triggered by pain generator(s) that can be driven by the peripheral nervous system or the CNS, psychologically driven, or a combination, and can be applied to a diverse range of clinical conditions that share common neurophysiological mechanisms and can involve various organ systems. However, a full understanding of the underlying mechanisms of nociplastic pain remains elusive. Hence, treating and caring for patients with nociplastic pain is challenging, with management strategies directed towards attenuating, rather than eradicating, symptoms, in addition to other treatments including improving function and other quality of life indicators.

It has been reported that most medications provide only the modest benefit and are often associated with adverse effects, which are more likely to occur in nociplastic conditions (Jason L. A. et al., Psychosom. Med. 2000; 62:655-63). Furthermore, although strategies such as acceptance commitment therapy, emotional expression, and psychodynamic psychotherapy are commonly recommended for treating chronic pain, the evidence for their effectiveness (and for adverse effects) is insubstantial (Williams A. C. C. et al., Cochrane Database Syst Rev. 2020; 8: CD007407), as is the case for many treatments for chronic pain. Nevertheless, nociplastic pain is known to respond to different therapies than nociceptive pain, with a decreased responsiveness to peripherally directed therapies such as anti-inflammatory drugs and opioids, surgery, or injections.

Accordingly, there remains an urgent need for new and alternative options that are effective for preventing or treating chronic pain, such as nociplastic pain, whilst limiting some of the adverse side effects seen with many other analgesics.

### SUMMARY

The present disclosure provides a composition including a lactic acid bacterium and a carrier thereof for preventing or treating chronic pain. In at least one embodiment, the lactic acid bacterium is Lactococcus lactis, deposited under DSMZ Accession No. DSM 27019. In at least one embodiment, the lactic acid bacterium is in a form selected from the group consisting of a culture, a concentrate, a paste, a liquid, a dried product, a diluted product, and a crushed product. In at least one embodiment, the dried product is a spray-dried powder, a freeze-dried powder, a vacuum-dried powder, or a drum-dried powder. In at least one embodiment, the lactic acid bacterium is in a form of live or heat-inactivated bacterium. In at least one embodiment, the composition of the present disclosure is a dietary composition or a pharmaceutical composition. In at least one embodiment, the lactic acid bacterium is orally administered to the subject. In at least one embodiment, the effective amount is at least 1×10⁶ CFU, at least 1×10⁷ CFU, at least 1×10⁸ CFU, at least 1×10⁹ CFU, at least 1×10¹⁰ CFU, or at least 1×10¹¹ CFU, including 5×10⁶ CFU, 5×10⁷ CFU, 5×10⁸ CFU, 2×10⁹ CFU, 3×10⁹ CFU, 4×10⁹ CFU, 5×10⁹ CFU, 6×10⁹ CFU, 7×10⁹ CFU, 8×10⁹ CFU, 9×10⁹ CFU, 2×10¹⁰ CFU, 3×10¹⁰ CFU, 4×10¹⁰ CFU, 5×10¹⁰ CFU, 6×10¹⁰ CFU, 7×10¹⁰ CFU, 8×10¹⁰ CFU, 9×10¹⁰ CFU, 2×10¹¹ CFU, 3×10¹¹ CFU, 4×10¹¹ CFU, 5×10¹¹ CFU, 6×10¹¹ CFU, 7×10¹¹ CFU, 8×10¹¹ CFU, and 9×10¹¹ CFU, but not limited thereto.

In at least one embodiment of the present disclosure, a method for preventing or treating a chronic pain in a subject in need thereof is also provided. In at least one embodiment, the method of the present disclosure comprises administering an effective amount of Lactococcus lactis DSM 27019 to the subject in need thereof.

In at least one embodiment, the chronic pain is a nociplastic pain. In at least one embodiment, the nociplastic pain is caused by fibromyalgia, irritable bowel syndrome, bladder pain syndrome, complex regional pain syndrome type 1, or temporomandibular disorder. In at least one embodiment, the nociplastic pain is a diffuse sensitization, a functional visceral pain, or a regional somatic sensitization. In at least one embodiment, the diffuse sensitization is a pain caused by fibromyalgia. In at least one embodiment, the functional visceral pain or the regional somatic sensitization is a pain caused by irritable bowel syndrome.

In at least one embodiment of the present disclosure, a method for reducing a neurotransmitter of pain thereby preventing or treating a chronic pain in a subject in need thereof is also provided, comprising administering an effective amount of Lactococcus lactis DSM 27019 to the subject in need thereof. In at least one embodiment, the neurotransmitter is substance P.

Also disclosed herein is a method of preventing or treating fibromyalgia in a subject in need thereof, comprising administering an effective amount of Lactococcus lactis DSM 27019 to the subject. In at least one embodiment of the present disclosure, the administration of Lactococcus lactis DSM 27019 reduces nociplastic pain of fibromyalgia in the subject. In at least one embodiment, the nociplastic pain of fibromyalgia is a diffuse sensitization. In at least one embodiment of the present disclosure, the administration of Lactococcus lactis DSM 27019 ameliorates depression in preventing or treating fibromyalgia in a subject in need thereof.

Also disclosed herein is a method of preventing or treating irritable bowel syndrome in a subject in need thereof, comprising administering an effective amount of Lactococcus lactis DSM 27019 to the subject. In at least one embodiment of the present disclosure, the administration of Lactococcus lactis DSM 27019 reduces nociplastic pain of irritable bowel syndrome in the subject. In at least one embodiment, the nociplastic pain of irritable bowel syndrome is a regional somatic sensitization. In at least one embodiment of the present disclosure, the administration of Lactococcus lactis DSM 27019 reduces a level of substance P in preventing or treating irritable bowel syndrome in a subject in need thereof. In at least one embodiment of the present disclosure, the administration of Lactococcus lactis DSM 27019 increases mucin production in preventing or treating irritable bowel syndrome in a subject in need thereof. In at least one embodiment of the present disclosure, the administration of Lactococcus lactis DSM 27019 promotes intestinal barrier function in preventing or treating irritable bowel syndrome in a subject in need thereof.

### BRIEF DESCRIPTIONS OF DRAWINGS

The present disclosure can be more understood by reading the following descriptions of the embodiments, with reference made to one or more of the accompanying drawings below.

FIG. 1 shows the plot of the paw withdrawal (PW) threshold to the mechanical stimuli over time in an animal model of fibromyalgia. *p<0.05, **p<0.01 relative to baseline (BL).

FIG. 2 shows the relative time of immobility in the forced swim test (FST) in an animal model of fibromyalgia. #p<0.05 compared with the MP group.

FIG. 3 shows the visceromotor responses before (baseline) and after the injection of 5-HTP in saline group and DSM 27109 group, respectively. **p<0.01, ***p<0.001 compared with the baseline.

FIG. 4 shows immunoreactivity level of substance Pin L6 and S1 spinal cord. RFU, relative fluorescence unit. *p<0.05 compared with the Ctrl group; ##p<0.01 compared with the saline group.

FIG. 5 shows the quantification of Alcian blue-positive area in distal colon section of the rats. **p<0.001 compared with the Ctrl group; ###p<0.001 compared with the saline group.

FIG. 6 shows the total fluorescence area of occludin and ZO-1 in the distal colon of the rats. *p<0.05 compared with the Ctrl group; ##p<0.001 compared with the saline group.

FIG. 7 shows the paw withdrawal threshold in response to mechanical stimuli at different time points in somatic hypersensitivity animal model treated with different strains of lactic acid bacteria. Error bars are expressed as means±standard deviation. *p<0.05, ***p<0.001 compared to the saline group.

### DETAILED DESCRIPTION

The following examples are used for illustrating the present disclosure. A person skilled in the art can easily conceive the other effects of the present disclosure, based on the disclosure of the specification. It will be apparent that one or more embodiments may be practiced without specific details. The present disclosure can also be implemented or applied as described in different examples. It is possible to modify or alter the following examples for carrying out this disclosure without contravening its scope for different applications. Titles or subtitles may be used in this disclosure for the convenience of a reader, which shall have no influence on the scope of the present disclosure.

In this disclosure, all terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein are defined based on the meaning of the terms together with the descriptions throughout the specification.

As used in this disclosure, the singular forms “a,” “an,” and “the” include plural referents unless expressly and unequivocally limited to one referent. The term “or” is used interchangeably with the term “and/or” unless the context clearly indicates otherwise.

Also, when a part “includes” or “comprises” a component or a step, unless there is a particular description contrary thereto, the part can further include other components or other steps, not excluding the others.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

The phrase “an effective amount” refers to the amount of an active ingredient that is required to result in a reduction, inhibition, or prevention of a chronic pain in a subject. An effective amount will vary, as recognized by those skilled in the art, depending on routes of administration, excipient usage, and the possibility of co-usage with other therapeutic treatment.

As used herein, the terms “subject” and “individual” may be interchangeable and refer to an animal, e.g., a mammal including the human species. The term “subject” is intended to refer to both the male and female gender unless one gender is specifically indicated. Non-limiting examples of non-human animal subjects include: rodents such as mice, rats, hamsters, and guinea pigs; rabbits; dogs; cats; sheep; pigs, goats; cattle; horses; and non-human primates such as apes and monkeys.

As used herein, the term “prophylactic,” “preventing,” or “prevention” refers to preventive or avoidance measures for a disease or symptoms or conditions of a disease, which include but are not limited to applying or administering one or more active agents to a subject who has not yet been diagnosed as a patient suffering from the disease or the symptoms or conditions of the disease but may be susceptible or prone to the disease, e.g., a chronic pain. The purpose of the preventive measures is to avoid, prevent, or postpone the occurrence of the disease or the symptoms or conditions of the disease.

As used herein, the term “treat,” “treating,” or “treatment” refers to the application or administration of one or more active agents to a subject afflicted with a disorder, a symptom or a condition of a disease, or a progression of the disease, with the purpose to cure, heal, relieve, alleviate, alter, remedy, ameliorate, improve, or affect the disorder, the symptom or the condition of the disease, the disabilities induced by the disease, or the progression of the disease.

As used herein, the term “administration” refers to the placement of an agent described herein, which is an effective amount of a lactic acid bacterium, into a subject by a method or route which results in at least partial localization of the agent at a desired site. An agent described herein can be administered by any appropriate route which results in effective treatment in the subject, i.e., administration results in delivery to a desired location in the subject where at least a portion of the agent delivered. Exemplary modes of administration include, but are not limited to, injection, infusion, instillation, or ingestion.

The present disclosure provides a lactic acid bacterium, a composition comprising the lactic acid bacterium, and a method for preventing or treating a chronic pain by administering the composition to a subject in need thereof. In the context of the present disclosure, the term “chronic pain” means, but is not limited to, a pain that persists more than one month, e.g., more than three months. As defined by the International Association for the Study of Pain (IASP), chronic pain is as an unpleasant sensory and emotional experience associated with, or resembling that associated with, actual or potential tissue damage. This definition acknowledges that pain can occur in the absence of identifiable tissue damage, such as in fibromyalgia. The three main categories of chronic pain include nociceptive, neuropathic, and nociplastic. Nociceptive pain results from activity in neural pathways, secondary to actual stimuli or stimuli that might potentially damage tissue. Nociceptive pain is the most common form of chronic pain, encompassing arthritis and most forms of spinal pain. Neuropathic pain is defined by the IASP as pain caused by damage or disease affecting the somatosensory nervous system. Compared with nociceptive pain, neuropathic pain is typically associated with sensory abnormalities, such as numbness and allodynia, more prominent pain paroxysms and, depending on the nerve(s) affected, neurological findings. Typical descriptors for nociceptive pain include terms such as aching and throbbing, whereas neuropathic pain is generally described with adjectives such as lancinating and shooting.

Approximately 15 to 25% of chronic pain is neuropathic, with the most common conditions including diabetic neuropathy, postherpetic neuralgia, and radiculopathy. As opposed to many forms of nociceptive pain and acute nerve injury, chronic neuropathic pain is always maladaptive. Nociplastic pain is pain that arises from the abnormal processing of pain signals without any clear evidence of tissue damage or discrete pathology involving the somatosensory system. Also known as functional pain syndromes, these conditions include pain states such as fibromyalgia, irritable bowel syndrome, and possibly non-specific back pain. The pathophysiological mechanisms that cause these disorders primarily involve augmented sensory processing and diminished inhibitory pathways. Nociplastic pain includes diffuse sensitization of fibromyalgia, functional visceral pain or visceral hypersensitivity in irritable bowel syndrome or bladder pain syndrome, regional somatic sensitization in complex regional pain syndrome type 1 or temporomandibular disorder, peripheral sensitization due to proliferation of sodium channels or sympatho-afferent coupling, central sensitization due to N-methyl-D-aspartate activation or cortical reorganization, diminished descending inhibition such as periaqueductal grey and rostroventromedial medulla, and immune system activation including glial cells, chemokines, cytokines, and other inflammatory mediators. Common nociplastic pain syndromes include chronic widespread pain, fibromyalgia, chronic low back pain of unknown causes, chronic temporomandibular pain disorders, irritable bowel syndrome, chronic primary bladder pain syndrome, and chronic primary pelvic pain syndrome.

### EXAMPLES

Exemplary embodiments of the present disclosure are further described in the following examples, which should not be construed to limit the scope of the present disclosure.

### Materials and Methods

### Preparation of Lactococcus Lactis DSM 27109

The isolated L. lactis DSM 27109 (hereinafter DSM 27109) was inoculated in de Man Rogosa Sharpe (MRS) broth (Criterion, Hardy Diagnostics, Santa Maria, CA, USA), cultured anaerobically at 37° C. for 18 h, and then harvested by centrifugation at 6,000×g for 10 min. The pellet was re-suspended in MRS plus 12.5% glycerol to a final concentration of 5×10¹⁰ colony-forming unit (CFU)/mL and then aliquoted and stored at −20° C. until use. Before oral administration to the rodents, the aliquot was pre-warmed to 37° C. for 1 h, centrifuged at 6,000×g for 10 min, discarded supernatant, and resuspended in phosphate buffered saline (PBS).

### Animals

C57BL/6J male mice aged 8 weeks were purchased from the National Laboratory Animal Center. Adult male Sprague-Dawley (SD) rats aged 8 weeks (300 to 400 g) were obtained from the National Yang Ming Chiao Tung University Laboratory Animal Center, Taipei, Taiwan. All animals were kept on a 12-hour light-dark cycle in a temperature-controlled environment (22±2° C.) with access to standardized laboratory chow and tap water ad libitum at the National Yang Ming Chiao Tung University Laboratory Animal Center. All protocols were approved by the Institutional Animal Care and Use Committee of National Yang Ming Chiao Tung University.

Mouse model of acid-induced muscle pain

The model of acid-induced muscle pain (MP) as reported in Lin Y. L., et al. (Elife, 2022 Nov. 15; 11: e78610) was used as a preclinical fibromyalgia-like MP model. Briefly, all mice were anesthetized, and the MP and control mice received 20 AL injections of acidic saline (pH 4.0) or neutral saline (pH 7.2), respectively, on day 0 in the left gastrocnemius muscle. After 3 days (day 3), the same gastrocnemius muscle was re-injected with acidic or neutral saline.

### Von Frey Filament Test

Mechanical hypersensitivity was evaluated using the von Frey filament test. A series of von Frey filaments with increasing stiffness (0.04 to 1.4 g) were applied to the plantar surface of both hind paws. Each filament was applied five times, and the lowest force that elicited at least three withdrawals out of the five stimuli was recorded as the threshold (g).

### Forced Swim Test

The forced swim test (FST) used a transparent acrylic cylindrical container (25 cm in height and 10 cm in width) filled with water to a depth of 16 cm. The mouse was placed in water maintained at 20° C. to 22° C. for 6 minutes. Immobility time, indicative of depressive behavior, was measured using the video tracking software Etho Vision XT 13 (Noldus Information Technology, Leesburg, VA, USA).

Colorectal distension electromyography recording of visceral hypersensitivity induced by 5-HTP injection

Colorectal distension (CRD) was performed with electromyography (EMG) recordings to evaluate the extent of visceral hypersensitivity (VH) as described in previous studies (Lu C. L. et al., Gastroenterology 137:1040-1050). First, Teflon-coated stainless steel EMG electrodes (7 strand, A-M Systems, Inc., Carlsborg, WA, USA) were implanted in the rats'abdominal external oblique muscle under anesthesia. The electrodes were then exteriorized at the back of the neck. Following electrode implantation surgery, the rats were given a two-week acclimation period. To acclimate the rats to the experimental conditions, they were placed alone in round, transparent plastic channel tunnels (6 cm in diameter and 25 cm in length) for 30 minutes per day for 3 days prior to the CRD experiments.

The CRD balloon was made from a latex glove finger (7 cm long) attached to a rectal catheter (Medtronic, Skovlunde, Denmark). To equilibrate the tension in its wall, the balloon was inflated and left overnight. During the CRD testing, the inflatable device was inserted through the anal canal into the rectum of conscious rats and secured at the base of the tail. The device was then connected to a barostat machine (Medtronic, Denmark). The colon was distended by inflating the balloon to the desired pressure (30, 60, or 80 mmHg) for 10-second intervals, with 30-second intervals between each distension. This process was repeated 4 times for each experimental protocol, with 5-minute intervals between each series. To assess the visceromotor response to CRD, EMG data were recorded using a CED 1401 instrument and analyzed with Spike 2 software (Cambridge Electronic Design, Cambridge, UK). The raw signal was rectified offline, and the area under the curve for baseline activities in each session was subtracted from the area under the curve for the rectified responses to CRD, yielding the difference between the areas under the curves. In each session, the EMG values from individual distensions were averaged.

After receiving a session of CRD at baseline, the rats rested for 30 minutes. Subsequently, 5-hydroxytryptophan (5-HTP, 5 mg/kg/rat) was subcutaneously injected into the saline group and DSM 27109 group to induce increased visceral hypersensitivity, while rats in the control group received a subcutaneous injection of saline (200 μL/rat). All rats rested for another 30 minutes and received a session of CRD to evaluate the extent of visceral hypersensitivity.

The CRD and 5-HTP injection in rats serves as an animal model of irritable bowel syndrome (IBS).

Immunofluorescence analysis of protein expression

Rats were deeply anesthetized and perfused with 10% formalin fixative (JT Baker, Center Valley, PA, USA). The L6-S1 spinal cord and distal colon were harvested, postfixed with 10% formalin at 4° C. for 4 hours, and dehydrated with 30% sucrose solution. These tissues were then embedded in OCT gel and sliced into 30 pm thick sections using a cryostat (CM1900, Leica, Germany).

The L6-S1 spinal cord sections were washed with tris-based saline containing 0.3% Triton X-100 (TBST), blocked with 5% skim milk for 2 hours, and incubated overnight with primary antibodies (rat anti-substance P, 1:200, GTX72999, GeneTex) at 4° C. Subsequently, the sections were washed with TBST and incubated with secondary antibodies (Rabbit anti-rat-FITC 647, 1:400, Jackson ImmunoResearch) in the dark for 2 hours. Finally, the sections were washed with TBST, covered with Fluoromount-G Mounting Medium (Invitrogen), and stored at 4° C. Fluorescent signals were detected using a confocal laser scanning microscope (FV10i, Olympus, Tokyo, Japan), and images were analyzed using the MetaMorph software.

The distal colon sections were washed in PBS containing 0.25% Triton X-100 (PBST), blocked with 1% bovine serum albumin and 5% normal goat serum for 1 hour at room temperature, and incubated overnight with primary antibodies (Rabbit anti-ZO-1 antibody, 1:50, Invitrogen; Rabbit anti-occludin antibody, 1:25, Proteintech) at 4° C. Subsequently, the sections were washed with PBST and incubated with secondary antibodies (Goat anti-rabbit-FITC antibody, 1:400, Millipore) in the dark for 2 hours. Finally, the sections were stained with Hoechst 33258 (1:2000) for 20 minutes, washed with PBS, covered with Fluoromount-G Mounting Medium, and stored at 4° C. Fluorescent signals were detected using a confocal laser scanning microscope (LSM700, Zeiss, Germany), and images were analyzed using MetaMorph software.

### Histological Analysis

The paraffin-embedded distal colon blocks were sectioned into 5-μm-thick slices. After deparaffinization and rehydration, the tissue sections were stained with Alcian Blue (pH 2.5), mounted on slides, and visualized under a microscope.

### Data Analysis and Statistics

Data were analyzed using GraphPad Prism 6 (GraphPad Software, Inc., San Diego, CA, USA). Statistical significance was tested using the one-way analysis of variance (ANOVA) with the Tukey's post hoc test or Mann-Whitney test at the significance level (p) indicated. Data are presented as mean±standard error of mean (SEM).

### Example 1. Lactococcus lactis DSM 27109 Alleviates Chronic Pain and Depression in Fibromyalgia Animal Model

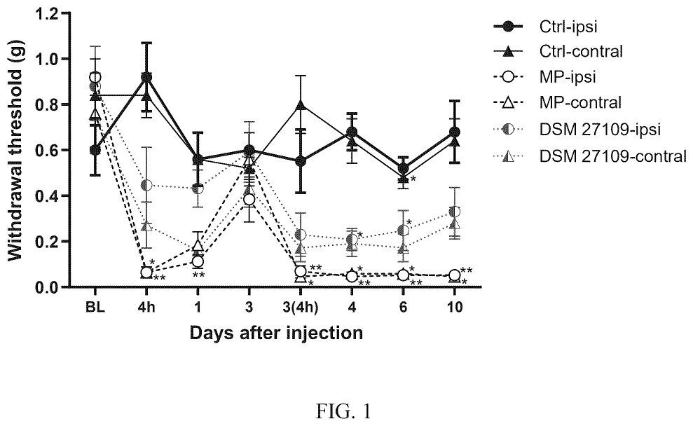

Acid-induced MP in rats or mice is considered a preclinical model of fibromyalgia (Sluka K. A. et al., Communicative & Integrative Biology 4:394-39). In this example, MP was induced in mice using a well-established acid-induced protocol as reported in the literature. Then, in the MP mice group, acidic saline (pH 4.0) was injected unilaterally into the gastrocnemius muscle on day 0 (baseline, BL) and day 3. Following the first acidic saline injection, the MP mice showed a transient decrease in the paw withdrawal (PW) threshold in both the ipsilateral (‘ipsi’) and contralateral (‘contral’) hind limbs in response to the von Frey filament stimulation, as shown in FIG. 1. The acute pain mostly recovered by day 3. However, a second injection caused a sustained decrease in the PW threshold that persisted for at least 10 days. In contrast, control mice (Ctrl) injected with neutral saline (pH 7.2) showed no significant changes in PW response to von Frey filament stimulation compared to MP mice.

Compared to the MP mice, those in the DSM 27109 group, which received oral administration of DSM 27109 (1×10⁹ CFU/mice/day) for 7 days prior to baseline, showed an increased PW threshold following the injection of acidic saline (FIG. 1). This suggests that DSM 27109 alleviates acid-induced MP.

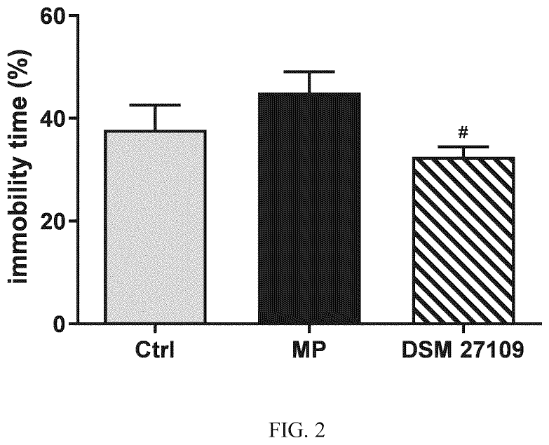

In addition to mechanical allodynia, mice with MP exhibited various affective symptoms, including depression. The FST, commonly used to assess depression-like behavior in mice, showed that MP mice spent more time immobile compared to control mice, as depicted in FIG. 2, indicating increased depression-like behavior. However, treatment with DSM 27109 reduced the immobility time (FIG. 2), suggesting that DSM 27109 could alleviate depression-like behavior in the MP mouse model.

### Example 2. Lactococcus lactis DSM 27109 Reduces Visceral Hypersensitivity (VH)

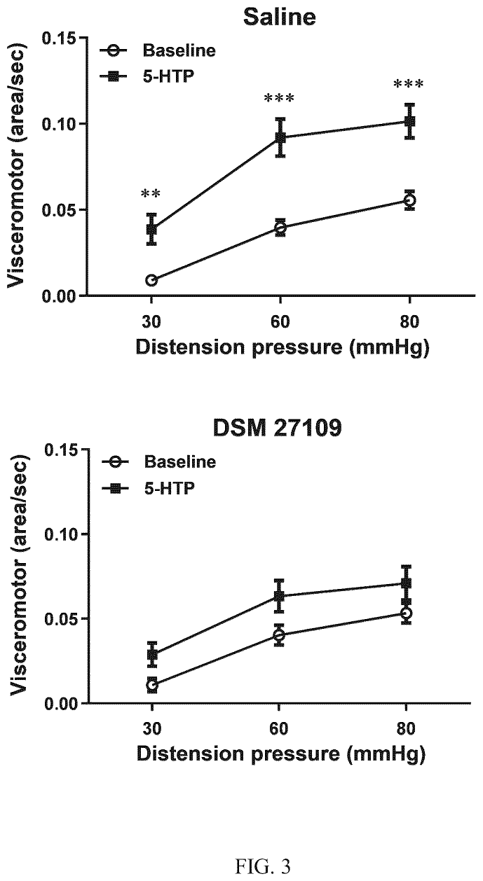

Colorectal distension (CRD) was performed to evaluate the effects of DSM 27109 in rats with 5-HTP-induced visceral hypersensitivity (VH). As shown in FIG. 3, the visceromotor reflex levels were comparable between the saline group and the DSM 27109 group at baseline (BL). In the saline group, 5-HTP injection significantly increased the visceromotor reflex levels at three distension pressures compared to baseline levels, also shown in FIG. 3. However, in the DSM 27109 group, which received oral administration of DSM 27109 (1×10¹⁰ CFU/rat/day) for 14 days prior to CRD, no significant increase was observed. These results suggest that DSM 27109 ameliorates 5-HTP-induced VH in rats.

### Example 3. Lactococcus lactis DSM 27109 Reduces Substance P (Sp) Level

SP is a neurotransmitter associated with the transmission of pain information to the spinal cord and brain. Previous study has revealed that visceral pain signals in CRD-treated mice are primarily transmitted to the L6-SI segments of the spinal cord (Kyloh, M. et al., Front Neurosci. 5:16). To further confirm the effect of DSM 27109 on 5-HTP-induced VH, immunofluorescence staining was carried out to measure SP levels in the L6-S1 segments of the spinal cord.

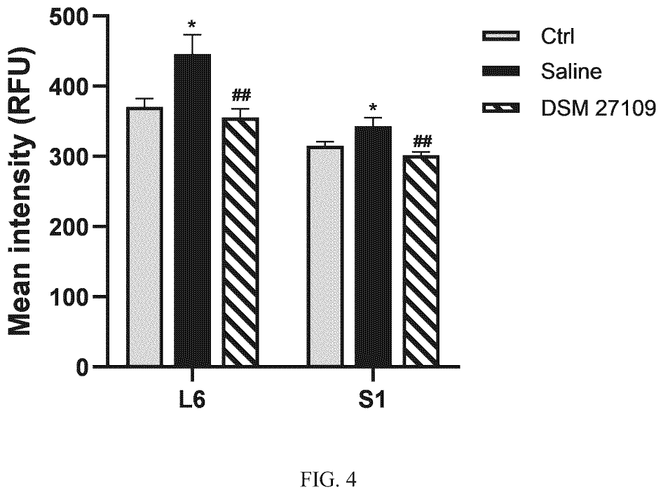

As a result, in both the L6 and S1 spinal cord segments, the immunoreactive signal of SP was higher in the saline group compared to the control group, while administration of DSM 27109 reduced the signal of SP (FIG. 4), suggesting that DSM 27109 can decrease SP levels in rats with 5-HTP-induced VH.

### Example 4. Lactococcus lactis DSM 27109 Increases Mucin Production and Strengthens Colon Barrier

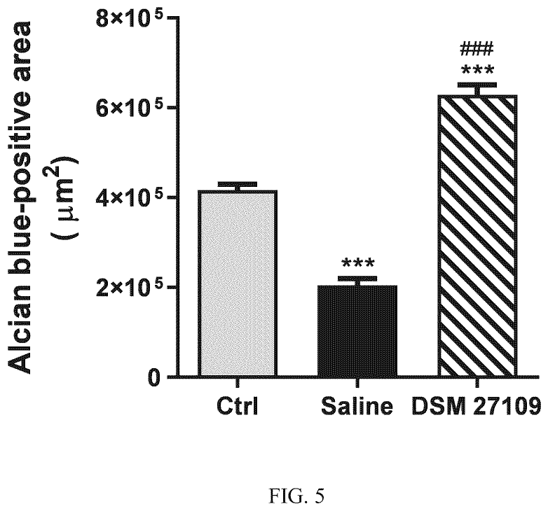

Mucins are major components of the colonic mucus barrier. To investigate whether DSM 27109 supplementation affects mucin expression in a rat model of 5-HTP-induced IBS, Alcian blue staining was performed to analyze mucin production in the distal colon. FIG. 5 shows the quantification of the Alcian blue-positive area in the colon sections. Compared to the control group, the saline group exhibited a smaller positive colonic mucin area, suggesting that 5-HTP injection damages the intestinal mucosal barrier. However, DSM 27109 supplementation markedly restored colonic mucin expression. As shown in FIG. 5, the mucin-containing area was significantly larger in the DSM 27109 group compared to the control group.

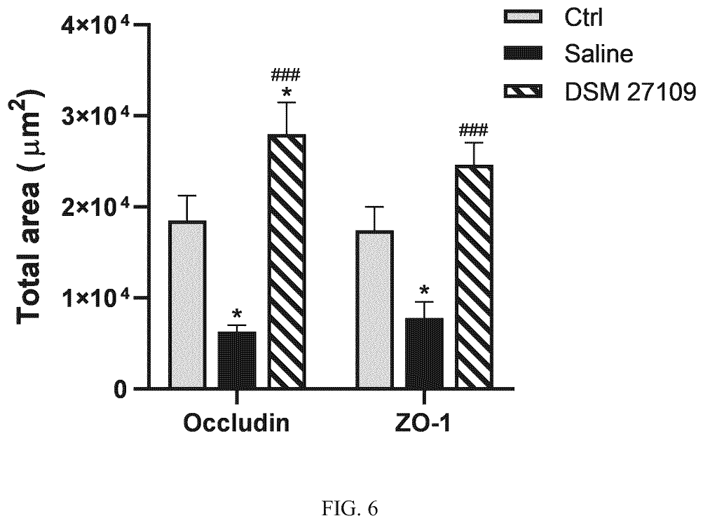

Tight junction proteins, such as occludin and ZO-1, maintain the intestinal barrier and regulate ion, nutrient, and water permeability. To assess intestinal permeability, the expression of occludin and ZO-1 in the colonic mucosa was analyzed using immunofluorescence staining. The fluorescence areas of the occludin and ZO-1 proteins were significantly lower in the saline group compared to the control group, as depicted in FIG. 6. After 14 days of DSM 27109 supplementation, the fluorescence areas of occludin and ZO-1 were elevated and were significantly higher than those in the saline group. Collectively, these data suggest that DSM 27109 supplementation promotes the expression and distribution of tight junction proteins, thereby maintaining proper intestinal barrier.

### Example 6. Lactococcus lactis DSM 27109 Reduces Somatic Hypersensitivity

To evaluate somatic hypersensitivity, an animal model of rats treated with intracolonic trinitrobenzene sulfonic acid (TNBS) was adopted. Previous research demonstrated that somatic hypersensitivity persists for at least 16 weeks following TNBS-induced colon inflammation. Briefly, 20 mg of TNBS in 50% ethanol (total volume, 0.4 mL) was instilled into the lumen of the colon, 3 to 4 cm proximal to the anus, using a 24-gauge catheter inserted 5 to 6 cm. Control rats received an equivalent volume of saline. Fourteen days after TNBS administration, the treated rats received either saline or different strains of lactic acid bacteria (DSM 27109, 836S, or 265P, 1×10⁹ CFU/day) daily from day 14 to day 28, while control rats were given saline orally. Somatic pain testing using Von Frey filaments was performed on days 14, 21, and 28 following intracolonic treatment with TNBS or saline.

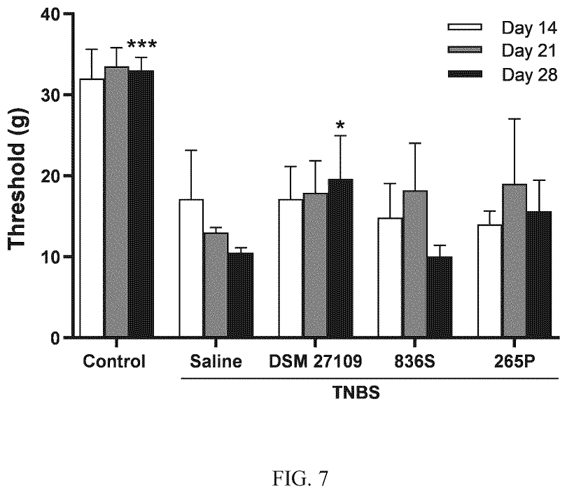

To identify the most effective lactic acid bacterial strain for relieving somatic pain, the TNBS model was used to test several strains. As shown in FIG. 7, rats are sensitive to mechanical stimulation after intracolonic treatment with TNBS. However, the treatment with lactic acid bacteria strain DSM 27109 increased the threshold of somatic hypersensitivity 28 days after the TNBS treatment, while the other two strains did not exhibit this effect. This suggests that DSM 27109 has the potential to increase the threshold of somatic hypersensitivity and alleviate pain.

While some of the embodiments of the present disclosure have been described in detail in the above, it is, however, possible for those of ordinary skill in the art to make various modifications and changes to the embodiments shown without substantially departing from the teaching of the present disclosure. Such modifications and changes are encompassed in the scope of the present disclosure as set forth in the appended claims.

What is claimed is:

1. A method for preventing or treating chronic pain in a subject in need thereof, comprising administering an effective amount of Lactococcus lactis DSM 27019 to the subject.

2. The method of claim 1, wherein the chronic pain is a nociplastic pain.

3. The method of claim 2, wherein the nociplastic pain is caused by fibromyalgia, irritable bowel syndrome, bladder pain syndrome, complex regional pain syndrome type 1, or temporomandibular disorder.

4. The method of claim 2, wherein the nociplastic pain is a diffuse sensitization, a functional visceral pain, or a regional somatic sensitization.

5. The method of claim 4, wherein the diffuse sensitization is a pain caused by fibromyalgia.

6. The method of claim 4, wherein the functional visceral pain or the regional somatic sensitization is a pain caused by irritable bowel syndrome.

7. The method of claim 1, wherein after the administration, at least one of a neurotransmitter of pain is reduced.

8. The method of claim 7, wherein the neurotransmitter is substance P.

9. A method of preventing or treating fibromyalgia in a subject in need thereof, comprising administering an effective amount of Lactococcus lactis DSM 27019 to the subject.

10. The method of claim 9, wherein the administration reduces nociplastic pain of fibromyalgia in the subject.

11. The method of claim 10, wherein the nociplastic pain is a diffuse sensitization.

12. The method of claim 9, wherein the administration ameliorates depression in the subject.

13. A method of preventing or treating irritable bowel syndrome in a subject in need thereof, comprising administering an effective amount of Lactococcus lactis DSM 27019 to the subject.

14. The method of claim 13, wherein the administration reduces nociplastic pain of irritable bowel syndrome in the subject.

15. The method of claim 14, wherein the nociplastic pain is a regional somatic sensitization.

16. The method of claim 13, wherein the administration reduces a level of substance P in the subject.

17. The method of claim 13, wherein the administration increases mucin production in the subject.

18. The method of claim 13, wherein the administration promotes intestinal barrier function in the subject.
